(19) United States
(12) Patent Application Publication
(10) Pub. No.: US 2011/0086828 A1
(43) Pub. Date: Apr. 14, 2011

(54) TREATMENT OF ALCOHOL ABUSE AND ALCOHOLISM USING MODULATORS OF NEUROSTEROID BINDING SITES ON GABAA RECEPTORS

(75) Inventor: Peter Winsauer, New Orleans, LA (US)

(73) Assignee: Board of Supervisors of Louisiana State University and Agricultural and Mechanical College, Baton Rouge, LA (US)

(21) Appl. No.: 12/900,192

(22) Filed: Oct. 7, 2010

Related U.S. Application Data
(60) Provisional application No. 61/278,424, filed on Oct. 7, 2009.

(51) Int. Cl. A61K 31/5685; A61K 31/56; A61P 25/32

(52) U.S. Cl. 514/179; 514/169

(57) ABSTRACT

The present invention provides methods for reducing alcohol drinking behavior in humans and treating acute alcohol poisoning through the use of neutral or negative modulating agents of the neurosteroid sites on GABA_{A} receptors. These agents avoid the unwanted side-effects of opiate antagonists and displays specificity toward the neurosteroid binding sites on GABA_{A} receptors.

### CROSS-REFERENCE TO RELATED APPLICATIONS

The present application claims the benefit of prior U.S. Provisional Application No. 61/278,424, filed Oct. 7, 2009, which is hereby incorporated by reference.

### FIELD OF THE INVENTION

The present invention relates generally to methods for the treatment of alcoholism and acute alcohol poisoning in humans, and in particular, to methods for the treatment of alcoholism and acute alcohol poisoning in humans through the administration of neutral or negative modulating agents of neurosteroid binding sites on GABA_{A} receptors.

### BACKGROUND

On an international scale, the World Health Organization estimates that alcohol dependence is the third leading cause of disease in developing countries worldwide. Alcohol use disorders, including alcohol abuse and dependence, affect seven to eight percent of Americans (about 15 to 20 million adults) at any given time. Accordingly, in the United States alone alcohol use disorders account for roughly $185 billion annually in healthcare costs, lost wages, bodily injury, and property damage.

Important advances have been made in the development of new drugs to treat alcoholism. There are currently several FDA-approved drugs which demonstrate some evidence of effectiveness in patients with alcohol dependence, including acamprosate, disulfram, and naltrexone, while several more drugs remain in the experimental stages, including bromocriptine, buspirone, carbamazepine, γ-hydroxybutyric acid (GHB), nalmefene, serotonin-specific reuptake inhibitors (SSRIs), and tricyclic antidepressants. Notwithstanding these advances, alcohol-dependent individuals represent a heterogeneous group, and, thus, a variety of pharmacological regimens are required for the effective treatment of alcoholics.

The current drugs on the market (e.g., opiate antagonists and acetaldehyde inhibitors such as disulfram) exhibit certain unwanted adverse effects and non-targeted or off-target toxicity, rendering them ineffective in many patients. Therefore, there is an unmet need for improved compositions and methods for the treatment of alcoholism. The ideal treatment method would be safe and effective with limited side effects.

### SUMMARY OF THE INVENTION

The present invention addresses many of the issues described above and provides methods for reducing alcohol drinking behavior in humans and treating accute alcohol poisoning. Agents utilized in embodiments of the present invention are neutral or negative modulators of the neurosteroid sites on γ-aminobutyric acid (hereinafter “GABA_{A}”)receptors. These agents avoid the unwanted side-effects of opiate antagonists and display specificity toward the neurosteroid binding sites on GABA_{A} receptors.

More particularly, according to an embodiment of the present invention a method for therapeutic treatment of alcoholism comprising administration of a modulating agent is provided that demonstrates specificity to neurosteroid binding sites on GABA_{A} receptor complexes. The modulating agent may be a neutral or negative modulator of the GABA_{A} receptor complex, and may include androstane, dehydroepiandrosterone (hereinafter “DHEA”), dehydroepiandrosterone sulfate (hereinafter “DHEA-S”), 3-acetyl-7-oxo-dehydroepiandrosterone (hereinafter “7-keto DHEA”), or analogs thereof. The modulating agent may also include a ligand that binds to at least one neurosteroid site on the GABA_{A} receptor complex.

In another example though non-limiting embodiment, the present inventive method is used to treat acute alcohol poisoning.

### BRIEF DESCRIPTION OF THE DRAWINGS

FIG. 1 Depicts a graph demonstrating preference for different concentrations of alcohol by rats who were subjected to either saline or alcohol during their adolescent phase of development.

FIG. 2 Depicts a graph demonstrating preference of rats for ethanol who were subjected to either saline or alcohol during adolescent development.

FIG. 3 Depicts a graph comparing the preference of rats for ethanol intake following food deprivation in rats subjected to either saline or alcohol during adolescent development.

FIG. 4 Depicts a graph demonstrating the effect of pregnanolone on alcohol intake in rats subjected to either saline or alcohol during adolescent development.

FIG. 5 Depicts a graph demonstrating the effect of DHEA on alcohol intake in rats subjected to either saline or alcohol during adolescent development, according to an exemplary embodiment of the present invention.

FIG. 6 Depicts a graph showing the effect of different concentrations of alcohol on rats subjected to either saline or alcohol during adolescent development.

FIG. 7 Depicts a graph comparing the effect of DHEA to 7-keto-DHEA in rats subjected to either saline or alcohol during adolescent development, according to an exemplary embodiment of the present invention.

### DETAILED DESCRIPTION OF THE INVENTION

The present invention provides methods for reducing alcohol drinking behavior in humans exhibiting alcoholism and for treating acute alcohol poisoning. The agents utilized in the methods of the invention are neutral or negative modulators of the neurosteroid sites on GABA_{A} receptors. These agents avoid the unwanted side-effects of opiate antagonists and display specificity toward the neurosteroid binding sites on GABA_{A} receptors.

It is noted that the administration of certain neuroactive steroids to humans may provide benefits to those with certain psychiatric disorders, such as stress disorders, anxiety disorders, and depression. See U.S. Patent Application Publication No. 2008/0070879 A1, which is hereby incorporated by reference.

According to an exemplary though non-limiting embodiment of the present invention, a method for therapeutic treatment of alcoholism may comprise administration of a modulating agent that demonstrates specificity to neurosteroid binding sites on GABA_{A} receptor complexes. The modulating agent may be a neutral or negative modulator of the GABA_{A} receptor complex, and may include androstane, DHEA, DHEA-S, 7-keto DHEA, or analogs thereof. The modulating agent may also include a ligand that binds to at least one neurosteroid site on the GABA_{A} receptor complex.

Neuroactive steroids can regulate some effects of ethanol. These steroids could regulate the mechanism of action of alcohol and serve as new pharmacotherapies for alcohol abuse. The interaction between neuroactive steroids and alcohol is important because both classes of drugs share similar effects. Mechanistically, for example, neurosteroids and alcohol bind to specific, yet separate, sites on GABA_{A} receptors and allosterically alter chloride flux similar to the barbiturates and benzodiazepines. In addition, both neurosteroids and alcohol bind to other ion channels and can allosterically alter their function.

Behaviorally, neuroactive steroids that act as positive modulators at GABA_{A} receptors and alcohol can produce anxiolytic, hypnotic, anticonvulsant and amnestic effects similar to one another while also substituting for one another in operant drug-discrimination procedures. Nevertheless, there are differences between the different classes of drugs that allosterically modulate GABA_{A} receptors and some of these differences translate to differences in their behavioral effects. Ethanol is often used illicitly in combination with other positive allosteric modulators of GABA_{A} receptors (suggesting additive reinforcing effects), but ethanol and other positive modulators do not necessarily decrease ethanol intake when combined.

Antagonists of GABA_{A} receptors such as picrotoxin and some drugs that can negatively modulate GABA_{A} receptors such as the imidazobenzodiazepine RO15—4513 can decrease ethanol intake. With regard to the effects of positive GABA_{A} modulators, the behavioral effects of the different classes of negative modulators are not always alike or consistent across studies, particularly with respect to their capacity for blocking ethanol intake, and the specific mechanism of action of these drugs is unknown.

Accordingly, in developing a method for the treatment of alcoholism using modulators of neurosteroid binding cites on GABA_{A} receptors, the effects of pregnanolone (a positive modulator of GABA_{A} receptors) and dehydroepiandrosterone (a negative modulator of GABA_{A} receptors) on ethanol intake were examined in twenty-two male Long-Evans hooded rats. While pregnanolone has no apparent effect on ethanol intake (except at the highest dose, which also produced sedative effects), dehydroepiandrosterone (DHEA) demonstrated the potential to markedly reduce oral self administration of ethanol.

In order to demonstrate methods for the treatment of alcoholism in accordance with the present invention, twenty-two male Long-Evans hooded rats were purchased at 25 days of age and served as subjects. Upon arrival, these subjects were housed 4 per cage and provided a diet of standard rodent chow ad libitum (Rodent Diet 5001, PMI Inc., St. Louis, Mo., USA) until postnatal day (hereinafter “PD”) 70. From PD 71 forward, the subjects were housed individually and maintained at 95% of their free-feeding weight during which a saccharin-fading procedure was conducted. Water was provided ad libitum in the home cage except during the experimental sessions. The home cage was made of polypropylene plastic and contained hardwood chip bedding. The colony room was maintained at 21±2 C.° with 50±10% relative humidity on a 14L:10D (light/dark) cycle (lights on 06:00 hours (h); lights off 20:00 h). Following training under the saccharin-fading procedure, the subjects were returned to ad libitum feeding conditions for 73 (minimum) to 89 (maximum) days during which an ethanol concentration-effect curve was established. After this curve was obtained, subjects were maintained at 95% of their free-feeding weight for the rest of the study. Ethanol preference training and experimental sessions were conducted daily during the light cycle between the hours of 12:00 h and 14:00 h. Subjects used in these studies were maintained in accordance with the Institutional Animal Care and Use Committee, Louisiana State University Health Sciences Center, and in compliance with the guidelines of the National Institute of Health Guide for Care and Use of Laboratory Animal Resources (publication No. 85-23, revised 1996).

### Adolescent Ethanol and Saline Administration

While still housed 4 per cage, subjects were randomly divided into two groups, a group that received ethanol between PD 35 and 63 (adolescent ethanol group) and a group that received saline during the same postnatal period (adolescent saline group). The adolescent ethanol group (n=11) received 2 g/kg of ethanol intraperitoneally (hereinafter “i.p.”) every other day, whereas the adolescent saline group (n=11) received an equal volume of saline every other day, for a total of 15 injections.

### Acquisition of Ethanol Drinking

Subjects were trained to consume ethanol orally using a modified saccharin/ethanol fading procedure (Samson, 1986; Leonard et al., 2006) starting on PD 75. Prior to each daily training session, animals were weighed and then returned to their home cage. Water bottles were removed and replaced for 30 min with 50-ml plastic centrifuge tubes containing a saccharin/ethanol solution and fitted with a rubber stopper and metal sipper tube. At the end of the 30-min session, the drinking tubes were removed, water bottles were replaced, and the volume of the solution consumed was determined by weighing the tubes (i.e., [(weight prior to the session—weight after the session)-0.4 ml)]. All solutions were prepared fresh daily. Rats initially received a 0.2% sodium saccharin(weight (g of sodium saccharin)/volume (100 mL solution))/0% ethanol (volume (mL of alcohol)/volume (100 mL solution)) solution that was gradually replaced until a solution of 0% sodium saccharin/10% ethanol was achieved. After a subject acquired stable saccharin/ethanol intake (±20% of the mean for 3 consecutive days) or a maximum of 8 days elapsed, the next sodiumsaccharin/ethanol solution was presented. Solutions were presented in the following order: 0.2% saccharin/0% ethanol, 0.15% saccharin/0.5% ethanol, 0.125% saccharin/1% ethanol, 0.1% saccharin/2% ethanol, 0.05% saccharin/5% ethanol, 0.01% saccharin/8% ethanol, and 0% saccharin/10% ethanol. Training, which took 40-57 sessions, and all subsequent testing were conducted 7 days per week.

### Ethanol Preference Test

After a stable baseline of ethanol drinking was established with a 10% ethanol solution, a concentration-effect curve for ethanol and water consumption was established using the standard two-bottle preference test. During these tests, subjects were allowed simultaneous access to two drinking tubes daily for 60 min in their home cage. One drinking tube contained tap water and the other contained varying concentrations of ethanol presented in the following order: 10%, 18%, 10%, 32%, 10%, 5.6%, 10%, 3.2%, 10% v/v. Immediately following the 60-min session, drinking tubes were removed, the water bottles were replaced, and the volume of water and ethanol consumed were determined. Each concentration of ethanol was presented daily until either stable consumption was observed (±20% of the mean for 3 consecutive days) or a maximum of 8 days had elapsed. As indicated above, after the criterion was met for each concentration, the subjects were always returned to the 10% ethanol concentration (baseline) until the above criterion was met. Data for consumption of 10% ethanol collected during different baselines were pooled. To avoid development of a positional bias, the positions of the drinking tubes were reversed each day. After completion of the ethanol concentration-effect curve under ad libitum feeding conditions, the subjects were again deprived to 95% of their free fed weight at that time, and the ethanol concentration-effect curve was redetermined. During the redetermination of the curve and all subsequent experimental manipulations, the preference sessions were decreased in duration from 60 to 30 minutes.

### Neuroactive Steroid Administration

Pregnanolone (5β-pregnan-3α-ol-20-one, Steraloids, Inc., Newport, R.I.) and dehydroepiandrosterone (DHEA; 5-androstene-3β-ol-17-one, Sigma-Aldrich, Inc, St. Louis, Mo.) were dissolved in a vehicle comprised of 45% (w/v) (2-hydroxypropyl)-γ-cyclodextrin (Sigma-Aldrich, Inc) and saline, and their effects on ethanol consumption were assessed using the two-bottle preference test as described above; however, the concentration of the ethanol solution that was presented daily was maintained at 18% (v/v) instead of 10%. Pregnanolone (110 mg/kg), DHEA (1-100 mg/kg) or vehicle were administered intraperitoneally 15 minutes prior to the daily 30-min session. Rats received the non-contingent injections of each dose daily until one of the criteria was met; that is, either a stable level of intake was observed (±20% of the mean for 3 consecutive days) or a maximum of 8 days had elapsed. After the testing of each dose was completed, subjects were always returned to the 18% ethanol concentration for the specified criterion. Doses of pregnanolone were administered in the following order: 1 mg/kg, 3.2 mg/g, vehicle, 10 mg/kg, 1.8 mg/kg, saline, and 5.6 mg/kg. Because DHEA's effect on ethanol consumption persisted some time after discontinuation of injections, injections of vehicle were interspersed in between doses of DHEA in the following order: 32 mg/kg, vehicle, 56 mg/kg, saline, 18 mg/kg, saline, 100 mg/kg, 1 mg/kg, vehicle, and 10 mg/kg. For pregnanolone and DHEA, the volume for both control (saline or vehicle) and drug injections was 0.1 ml/100 g body weight. To determine whether DHEA shifted the concentration-effect curve for ethanol, a concentration-effect curve for ethanol preference was also determined with and without 10, 32, and 56 mg/kg of DHEA as described above with a baseline concentration of ethanol maintained at 18%.

### Blood Collection and Blood Ethanol Concentration (BEC) Determinations

Blood samples were collected by saphenous venepuncture immediately after a 30-min two bottle preference test. Blood samples for four different ethanol concentrations were taken the day each subject met criteria. Serum was isolated and stored at −80° C. until blood ethanol levels (mg/dl) were quantified in duplicate using the MicroStat GM7 Analyzer (Analox Instruments, Inc., Lunenburg, Mass.). The intra-assay coefficient of variation was 2.5%.

### Data Analyses

Data for the volume of ethanol and water consumed were analyzed using two-way repeated measures analysis of variance (hereinafter “ANOVA”) (treatment condition×type of solution, and treatment condition×ethanol concentration) followed by Holm Sidak post-hoc tests when significant main effects were detected (SigmaStat Statistical Software, SYSTAT Software, Inc. Point Richmond, Calif., USA). The mean data for each subject were also grouped and analyzed for an effect of treatment at each dose of neurosteroid using a two-way repeated measures ANOVA (treatment condition×dose of neurosteroid). When a significant effect of treatment was detected, post-hoc Holm-Sidak tests were used to compare each dose with the respective control condition. Significance was accepted at a level of p<0.05 for all statistical tests.

### Results

### Adolescent Ethanol and Saline Administration

Prior to the initiation of ethanol and saline administration in the two groups of subjects on PD 35, the weights for the two groups were 146.64±1.88 g and 144.58±2.31 g (mean±standard error of mean (hereinafter “SEM”)), respectively. On PD 63 at the end of ethanol and saline administration, the weights for the two groups were 340.18±3.81 g and 331±5.14 g, respectively. When the grouped data were analyzed for an effect of treatment on body weight during the administration period, a two-way repeated measures ANOVA indicated that there was no effect of ethanol or saline treatment on body weights (F(1,20)=3.03, p>0.05), but there was a main effect of days due to the obvious weight gain during this period (F(25,500)=2573.66, p<0.001). There was no significant interaction (F(25,500)=1.02, p>0.05).

### Acquisition of Ethanol Drinking

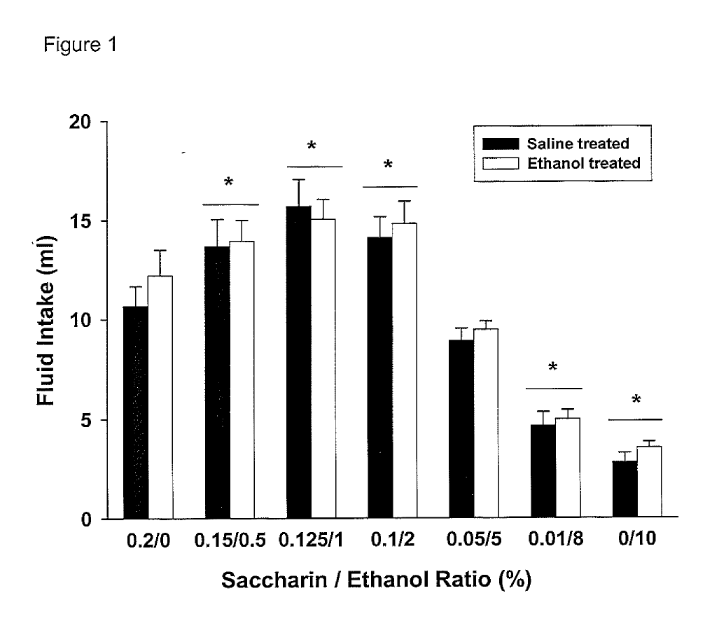

FIG. 1 illustrates the volume of intake in milliliters of varying solutions of sodium saccharin combined with ethanol during a saccharin-fading procedure. Solutions ranged from 0.2% saccharin with 0% ethanol (0.2/0) to 0% saccharin with 10% ethanol (0/10). Filled bars and vertical lines indicate mean and SEM for subjects that received saline as adolescents (n=11), whereas unfilled bars indicate mean and SEM for subjects that received ethanol as adolescents (n=11). Each solution was presented until one of two criteria were met; that is, until either intake did not vary by more than ±20% for 3 days or a total of 8 days, in which case the last 3 of those 8 days were averaged for comparability. Asterisks indicate significant differences for both adolescent-treated groups from saccharin alone (0.2/0) as there was no main effect of adolescent treatment or an interaction. A two-way repeated measures ANOVA indicated that there was a main effect of solution (F(6,120)=148.08, p<0.01), but no effect of adolescent ethanol or saline treatment on intake during training (F(1,20)=0.2, p>0.05) and no interaction (F(6,120)=0.698, p>0.05). As shown, the fading procedure produced an inverted U-shaped curve for ethanol consumption with intermediate solutions of saccharin and ethanol producing the highest intake and solutions with the largest concentrations of ethanol producing the lowest intake compared to a solution containing only saccharin (i.e., 0.2/0); however, intake of the final solution containing only 10% ethanol was well above zero for the subjects in both groups. More specifically, the mean intake was 2.78±0.49 mL for the group administered saline, and 3.53±0.31 mL for the group administered ethanol.

### Ethanol Preference and Intake Under Ad Libitum Conditions

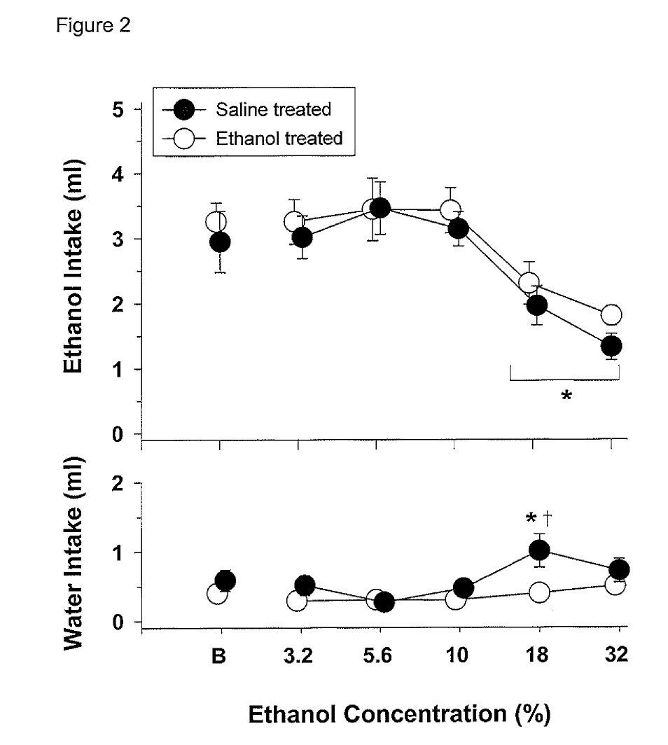

FIG. 2 shows the volume of ethanol (upper panel) or water (lower panel) intake in milliliters for increasing concentrations of ethanol during a two-bottle preference test with subjects fed ad libitum. Filled data points with vertical lines indicate mean and SEM for subjects that received saline as adolescents (n=11), whereas unfilled data points indicate mean and SEM for subjects that received ethanol as adolescents (n=11). Asterisks indicate significant differences from baseline (B) consumption of a 10% ethanol solution or water. The cross indicates a significant difference between the saline- and ethanol-treated adolescent groups. Ethanol was consumed preferentially over water at each of the five ethanol concentrations with preference ratios (ethanol intake/total fluid intake×100) ranging from 71.68% to 89.9%. A two-way repeated measures ANOVA conducted on the data for ethanol intake indicated that there was a significant effect of varying the ethanol concentration (F(5,100)=17.3, p<0.001), but no effect of adolescent saline or ethanol treatment on intake at the different ethanol concentrations (F(1,20)=0.62, p>0.05) or interaction of these factors (F(5,100)=0.21, p>0.05). Post hoc analyses of the effect of ethanol concentration indicated that both the 18 and 32% concentrations decreased intake in both groups. A similar two-way analysis of water intake indicated small differences compared to ethanol intake in that there was a significant effect of adolescent treatment (F(1,20)=7.1, p<0.05) and ethanol concentration (F(5,100)=3.73, p<0.05); however, this was due to a marked increase in water intake in the group that received saline as an adolescent when a 18% ethanol solution was presented. The interaction of these factors was not significant for water intake (F(5,100)=1.72, p>0.05).

Ethanol Intake Under 95% Food Deprivation and Blood Ethanol Concentrations

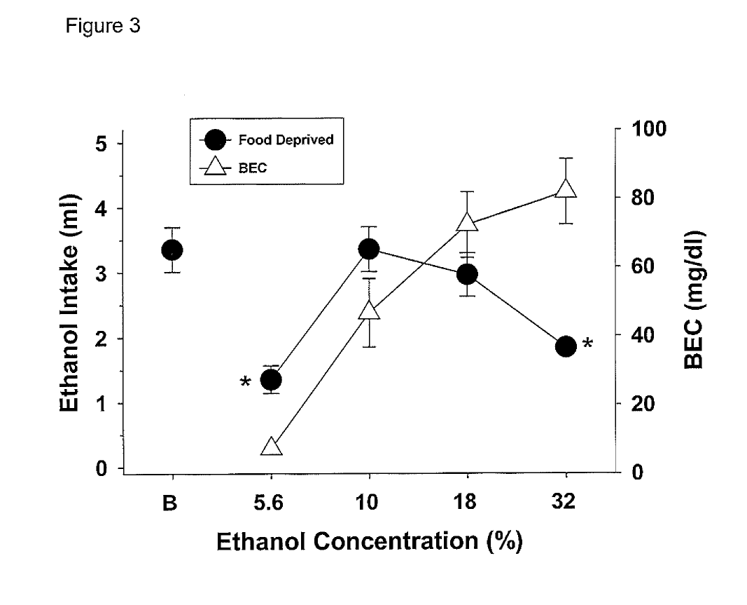

FIG. 3 shows the volume of ethanol intake in milliliters for increasing concentrations of ethanol during a two-bottle preference test with subjects under deprived feeding conditions (filled points, left axis). Groups of either saline- or ethanol-treated were combined for this concentration-effect curve as there was no main effect of adolescent treatment. The respective blood ethanol concentrations (hereinafter “BECs”) are also shown for the combined groups (unfilled triangles, right axis). Each concentration was presented until one of two criteria were met; that is, until either intake did not vary by more than ±20% for 3 days or a total of 8 days, in which case the last 3 of those 8 days were averaged for comparability. Blood to determine BEC was obtained on the final day of each concentration condition. Data points with vertical lines represent the mean and SEM. Asterisks indicate significant differences from baseline (“B”) consumption of a 10% ethanol solution. Water intake for the preference sessions in not shown as it was negligible. The groups were combined because a two-way repeated measures ANOVA indicated that there was no main effect of treatment (F(1,20)=0.004, p>0.05) and no interaction (F(3,60)=0.308, p>0.05) of adolescent treatment with ethanol concentration. Therefore, analyses of these data were comprised of a one-way ANOVA on ethanol concentration, which revealed a significant main effect of ethanol concentration on intake (F(3,63)=15.63, p<0.001) as depicted by an inverted U-shaped curve. Subsequent post-hoc tests also indicated that intake was reduced after substitution of the 5.6 and 32% ethanol concentrations compared to baseline. There was no significant effect of food deprivation on water consumption during the ethanol preference tests for either adolescent-treated group or at any ethanol concentration (data not shown). With regard to BEC, increasing concentrations of ethanol produced increasing BECs. Similar to the intake data, the BEC data were combined for these groups because no effect of adolescent treatment was apparent between groups.

### Effect of Pregnenolone on Ethanol Intake

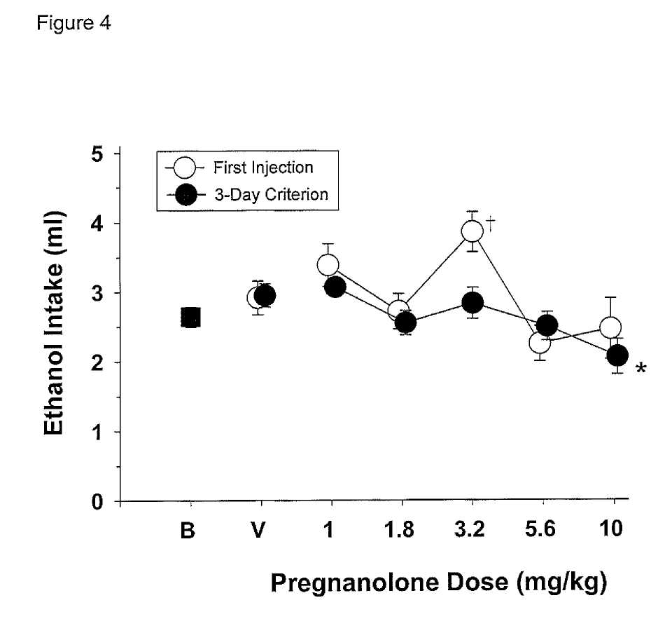

FIG. 4 illustrates the effects of increasing i.p. injections of pregnanolone on the intake of an 18% ethanol solution of rats (n=22) during 30-min preference sessions. Unfilled data points with vertical lines indicate the mean and SEM obtained for subjects on the first day of injection, whereas the filled data points with vertical lines indicate the mean and SEM obtained for subjects after they met the stability criterion (for additional details, see legend for FIG. 1). Asterisks indicate significant differences from sessions in which vehicle (“V”) was administered prior to the presentation of an 18% ethanol solution or water. The cross indicates a significant difference between the saline- and ethanol-treated adolescent groups. Baseline values are represented above “B”. Water intake for the preference sessions is not shown as it was negligible. The dose-effect curves shown reflect the mean intake data after the first day of injection with each dose of pregnanolone as well as the mean intake data after the subjects in each group met criterion (i.e., 3 days in which intake did not vary by more than 20% or the last 3 days of the 8-day maximum for each dose combination). The mean data in each curve again reflect the data for both adolescent treated groups combined as there was no difference between the groups. When a one-way ANOVA was conducted on the data for the first day of injection with each dose of pregnanolone tested, the analysis indicated that there was a significant effect of dose on ethanol intake (F(5,105)=4.96, p<0.01); however, none of the effects of dose were different from vehicle, rather the effects of the different doses on intake largely differed from each other in a non systematic way. For example, 3.2 mg/kg was significantly higher than 1.8, 5.6, and 10 mg/kg (p<0.05). In contrast, the mean data obtained for the subjects when the criteria were met for each dose were less variable and tended to be more orderly. There was a significant effect of dose (F(6,126)=5.02) and post-hoc analyses using the Holm-Sidak method indicated that the 10-mg/kg dose significantly differed from vehicle.

### Effect of DHEA on Ethanol Intake

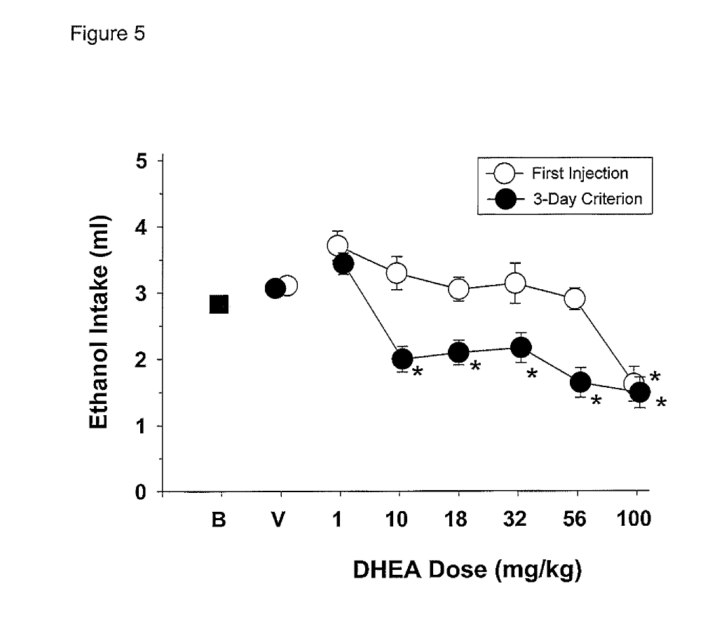

FIG. 5 demonstrates the effects of increasing doses of DHEA on the ethanol intake of rats (n=22) during 30-min preference sessions according to the preferred embodiment of the present invention. Unfilled data points with vertical lines indicate the mean and SEM obtained for subjects on the first day of injection, whereas the filled data points with vertical lines indicate the mean and SEM obtained for subjects after they met the stability criterion (for additional details, see legend for FIG. 4). As in the previous figure for pregnanolone, the data for both adolescent treated groups were combined as there were no significant main effects for this factor. With regard to the effects of DHEA, however, there was a significant effect for the highest dose of DHEA (100 mg/kg) in both groups on the first day of injection (F(6,126)=10.06, p<0.001) and for multiple doses after the criterion was met (F(7,147)=20,39, p<0.001). More specifically, post-hoc analyses of the mean data obtained after the stability criterion was met indicated that doses ranging from 10 to 100 mg/kg significantly decreased intake when compared to control injections of vehicle (p<0.05). Interestingly, ethanol intake also took 5 to 8 days to return to baseline levels after the final injection of each dose of DHEA (data not shown).

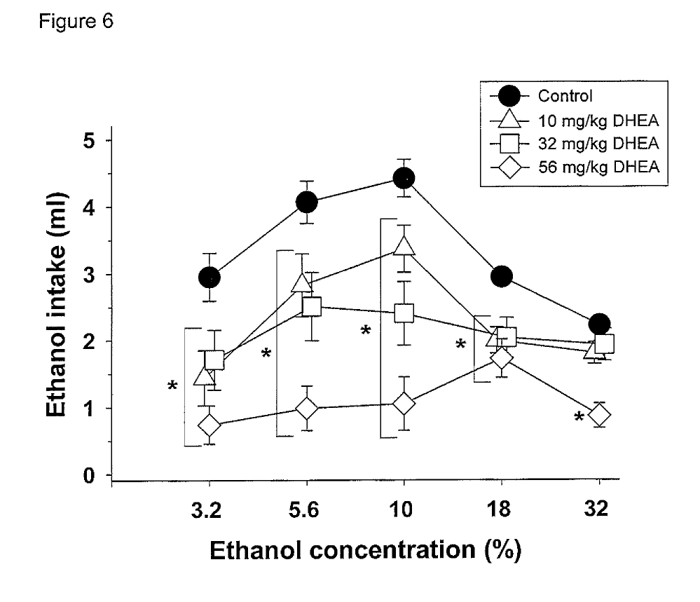

FIG. 6 shows the effects of i.p. injections of DHEA on the intake of rats (n=22) presented with increasing concentrations of ethanol during 30-min preference sessions, according to the preferred embodiment of the present invention. Filled data points with vertical lines (control) indicate the mean and SEM obtained for subjects when they were drinking 18% ethanol alone or 18% ethanol preceded by an injection of vehicle. Unfilled data points with vertical lines indicate the mean and SEM for the different doses of DHEA administered. Each dose of DHEA was administered with each concentration of ethanol until a stability criterion was met (for additional details, see legend for FIG. 1). Asterisks alone or asterisks with vertical brackets indicate significant differences from control conditions. Water intake for the preference sessions is not shown as it was negligible. As shown in FIG. 6, when 10, 32 and 56 mg/kg of DHEA were given in combination with varying concentrations of ethanol (3.2-32%), the ethanol-concentration effect curve was shifted downward significantly (i.e., there was a significant interaction between DHEA dose and ethanol concentration [F(12,420)=2.06, p<0.05)]). The effect of DHEA on ethanol intake was particularly evident after the 56-mg/kg dose of DHEA, which decreased intake of every concentration except the 18% concentration below 1 ml. The effects of the 10- and 32-mg/kg doses also decreased the intake of ethanol concentrations ranging from 3.2 and 18%; however, both of these doses produced comparable decreases in intake.

Effect of 7-Keto-DHEA on Ethanol Intake

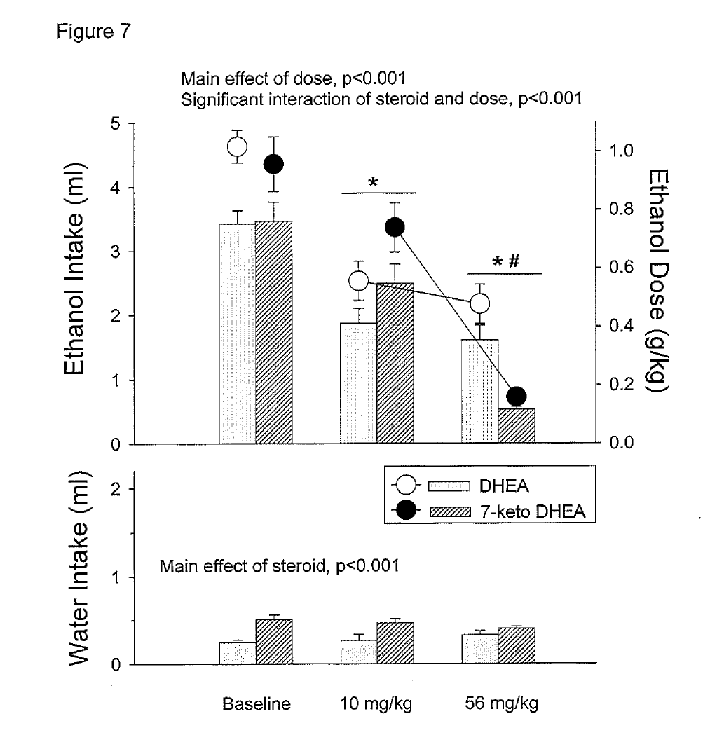

FIG. 7 compares the effects of two doses of DHEA with 7-keto-DHEA on the volume of ethanol or water intake (bars) and the dosage of ethanol (symbols) in rats (n=22) during 30-min preference sessions, according to exemplary embodiments of the present invention. 7-keto-DHEA is an analog of DHEA that is not converted to testosterone. Rats were administered either DHEA (n=22, unfilled points and unhatched bars) or 7-keto-DHEA (n=12-14, filled points and hatched bars) by i.p. injection 15 minutes prior to the session. The 18% concentration of ethanol (v/v) available during each preference session was presented alone or with each drug until one of two criteria were met; that is, until either intake did not vary by more than ±20% for 3 days or a total of 8 days, in which case the last 3 of those 8 days were averaged for comparability. As shown, both 10 and 56 mg/kg of DHEA and 7-keto-DHEA produced dose-dependent decreases in the volume and dosage of ethanol when compared to the respective baseline conditions. Water was not significantly different in DHEA or 7-keto-DHEA treated animals.

While a number of example embodiments of the present invention have been described, it is understood that these example embodiments are illustrative only, and not restrictive, and that many modifications would be apparent to those of ordinary skill in the art. Further still, any steps described herein may be carried out in any desired order, and any desired steps may be added or deleted. Further still, the use of DHEA as described herein is not limited to the treatment of alcoholism and may include, but is not limited to, treatment of acute alcohol poisoning, inhibition of atherosclerosis in humans, other primates, and rabbits, an anti-obesity steroid for humans, rats, and dogs, an immune stimulator in humans, mice, and rats, a neurosteroid for monkeys, rats and mice, and an anti-aging hormone for humans, mice, and rats.

What is claimed is:

1. A method for therapeutically treating alcoholism in humans, comprising: administration of an effective amount of a composition comprising a modulating agent in a pharmaceutically acceptable vehicle to an individual in need of treatment to reduce alcohol intake, wherein the modulating agent demonstrates specificity to the neurosteroid binding sites on GABA_{A} receptor complexes.

2. The method of claim 1, wherein the modulating agent is a neutral or negative modulator of the GABA_{A} receptor complex.

3. The method of claim 1, wherein the modulating agent is an androstane or an analog thereof.

4. The method of claim 1, wherein the modulating agent is dehydroepiandrosterone (DHEA) or an analog thereof.

5. The method of claim 1, wherein the modulating agent is dehydroepiandrosterone sulfate (DHEA-S) or an analog thereof.

6. The method of claim 1, wherein the modulating agent is 7-keto dehydroepiandrosterone (7-keto DHEA) or an analog thereof.

7. The method of claim 1, further comprising binding a modulating ligand to at least one neurosteroid site on the GABA_{A} receptor complex.

8. The method of claim 1, wherein the administration is oral, topical, or parenteral.

9. A method for therapeutically treating acute alcohol poisoning in humans, comprising: administration of an effective amount of a composition comprising a modulating agent in a pharmaceutically acceptable vehicle to an individual in need of treatment of acute alcohol poisoning, wherein the modulating agent demonstrates specificity to the neurosteroid binding sites on GABA_{A} receptor complexes.
